(19) United States
(12) Patent Application Publication
(10) Pub. No.: US 2008/0039942 A1
(43) Pub. Date: Feb. 14, 2008

(54) Spinal implant

(76) Inventor: Brian J. Bergeron, Austin, TX (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/503,473

(22) Filed: Aug. 11, 2006

(51) Int. Cl. A61F 2/44

(52) U.S. Cl. 623/17.16

(57) ABSTRACT

A spinal implant that includes a plurality of components adapted for use as an implant in a disc. Each component is configured to be inserted into a void of a disc and each component complimentarily abuts at least one other component to form the implant. A method treating a spinal disc that includes creating a port from a posterior approach into the disc space of a spinal disc; and inserting components that form a spinal implant into the disc space through the port to form the spinal implant. A method of forming a modular spinal implant, comprising: preparing a plurality of components that are shaped for insertion into a disc, wherein each component abuts at least one other component in the disc.

### FIELD OF THE INVENTION

This invention concerns a modular spinal implant for posterior insertion that includes a plurality of components made from an elastic material.

### BACKGROUND OF THE INVENTION

Spinal implants are frequently rigid and are surgically implanted by an anterior approach. Such invasive surgery may cause substantial scarring. Spinal implants have been proposed that are polymeric materials, setting up either in the disc space or presented as a monolithic structure. Such polymeric-based devices are also inserted through an anterior approach, which may lead to scarring.

Removal of prior devices can be complicated by ingrowth of bone, leading to potential cutting of vertebrae in order to remove an implant if it requires replacement.

The inventors have recognized that a need exists for a spinal implant that can be introduced into a disc space, such as after discectomy, through a minimally invasive posterior approach and that alleviates the prior problems discussed above.

### SUMMARY OF THE INVENTION

This invention provides a solution to one or more of the disadvantages discussed above.

The spinal implant of this invention may also be referred to as an artificial disc. The implant is made from a viscoelastic material, and is adapted for introduction into a disc space from a posterior approach, an extreme lateral approach, or a transforaminal approach. The implant is modular, being composed of a plurality of components, which together mate to form the implant.

In one broad respect, this invention is a multicomponent spinal implant, comprising: a plurality of components adapted for use as an implant in a disc space, wherein each component is configured to be inserted into the disc space and wherein the components interconnect within the disc to form the spinal implant. In one embodiment, each component complimentarily abuts at least one other component to form the implant. The components may interconnect by at least one link that is adapted to facilitate cinching of the components within the disc.

In another broad respect this invention is a multicomponent spinal implant, comprising: a plurality of components adapted for use as an implant in a disc space, wherein each component is configured to be inserted into the disc space, wherein the components interconnect within the disc to form the spinal implant, and wherein the components are interconnected by at least one link that is adapted to facilitate cinching of the components within the disc.

In another broad respect, this invention is a method of treating a spinal disc, comprising: creating a port from a posterior approach into the disc space of a spinal disc; and inserting components that form a spinal implant into the disc space through the port to form the spinal implant. In one embodiment, the disc has been subjected to a full discectomy prior to introduction of the implant. The method can optionally include repairing the tissue through which the port has been created.

In another broad respect, this invention is a method of forming a modular spinal implant, comprising: preparing a plurality of components that are shaped for insertion into a disc, wherein each component abuts at least one other component in the disc.

In one embodiment, a first component has a convex side surface and a second component has a concave side surface, which compliments the convex side of the first component. In this configuration, each component has a top, bottom, and at least one side, wherein the side has a surface that is not perpendicular to the surfaces of the top and bottom. In another embodiment, each component has a top, bottom, and at least one side, wherein the side has a surface generally perpendicular to the surfaces of the top and bottom. In one embodiment, when placed in the disc space the first and second components have a reduced range of motion relative to one another in a direction generally in line with the spine. In one embodiment, at least two components have complimentary flared sides to reduce motion when in the disc. In another embodiment, the components are shaped to pair together so as to limit range of motion relative to one another in a direction generally parallel to the spine. The implant is typically used after a discectomy to remove the nucleus pulposus. In general, the implant comprises components that align in a void in a disc after a discectomy to generally form a shape that substantially fills the void. The components compliment one another to thereby align to form the implant. The interaction between the components thus align, optionally interlock, and couple to form the implant. The implant of this invention can be referred to as a modular implant. In one embodiment, the components are inserted using a guidance member. In one embodiment, each component has at least one hole adapted for receipt of a guidance member. In one embodiment, the components include links that are cinched proximally to interconnect the components and form the spinal implant. In one embodiment, at least one component is anchored to a wall of the annulus opposite the port.

This invention has a number of advantages. First, the spinal implant of this invention is configured for insertion using a posterior approach during surgery. Relative to a typical surgery using an anterior approach, the present invention is substantially less invasive and will have less scarring. Secondly, the spinal implant mimics a healthy disc in that the implant is viscoelastic, thus enabling the spine to bend in a more normal fashion than a typical implant made from metals. In addition, the implant of this invention is relatively easy to remove if it needs to be replaced, and through practice of this invention, exit strategies are conserved. The modular implant of this invention is easier to remove than a one-piece elastomeric implant that is formed in site or which is introduced via an anterior approach. Likewise, implants positioned via an anterior approach frequently have substantial bone ingrowth that results in the need for cutting away bone if the implant is replaced, which cutting is obviated in the practice of this invention.

### BRIEF DESCRIPTION OF THE DRAWINGS

FIGS. 1A-1G show perspective views representative embodiments of the spinal implant of this invention.

FIGS. 2A-2C show representative top views of various configurations for the spinal implant of this invention.

FIGS. 3A-3K show representative views of additional embodiments of the spinal implant of this invention.

FIGS. 4A-4C show representative views of a spinal implant of this invention being placed in a disc.

FIG. 5A-5E show representative views of another embodiment of this invention being placed within a disc.

FIG. 6 shows a representative view of another embodiment of the spinal implant of this invention.

FIGS. 7A-7C show representative views of another spinal implant of this invention being placed within a disc.

FIG. 8. shows a representative view of a component being inserted into the disc space using a guidance tool.

FIG. 9 shows a representative view of components being inserted into a disc space through use of an anchor on the annulus wall opposite to the port.

FIG. 10 shows a representative view of another embodiment of the spinal implant of this invention

### DETAILED DESCRIPTION

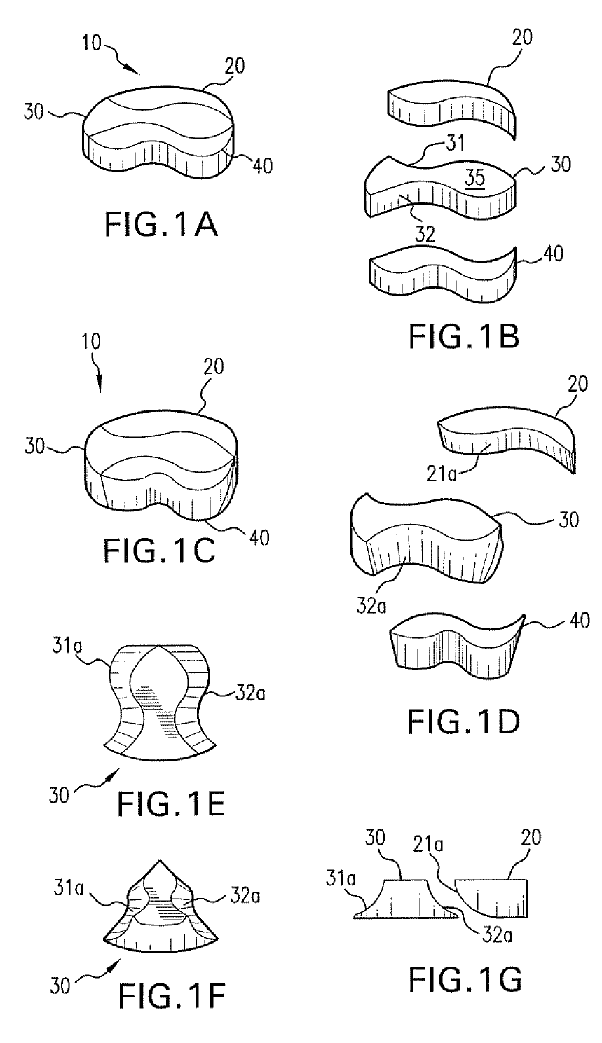

FIGS. 1A and 1B show one embodiment of the spinal implant of this invention. The implant 10 is composed of a first component 20, a second component 30, an a third component 40. Together the components form an implant 10 that is configured and sized to fit into a given spinal disc. The implant 10 is sized according to the disc where the implant is to be inserted. It should be appreciated that the components compliment one another, and when placed in the disc abut one another. In FIGS. 1A and 1B, the sides 31, 32 of the second component 30 are perpendicular to the face 35. The sides of the first component 20 and the third component 40 are similarly perpendicular to their respective faces. In FIGS. 1A and 1B, the second component 30 has a “fish” shaped face 35, with the components together forming an implant 10. The implant 10 can be configured with the sides of each component pointing in different directions depending on whether the implant is inserted via a posterior approach, an extreme lateral approach, or a transforaminal approach.

The implant 10 can alternatively have slanted or concave/convex sides that compliment one another at the locations where the components abut. The implant 10 depicted in FIGS. 1C and 1D includes a convex side 21a and a concave side 32a that complement one another so that the implant is generally locked in place so that transverse movement (in the direction of a spine) of the components is minimized within a disc. FIGS. 1E and 1F show the second piece 30 that illustrates the approximate, representative shape of the two sides 31a, 32a from a plan view and a side perspective view, respectively. FIG. 1G illustrates a side view of a first component 20 and a second component 30, and shows an example of convex side 21a and concave side 31a that compliment one another so that each component abuts the other along at least a portion of the implant, and preferably abuts along the entire length of the implant.

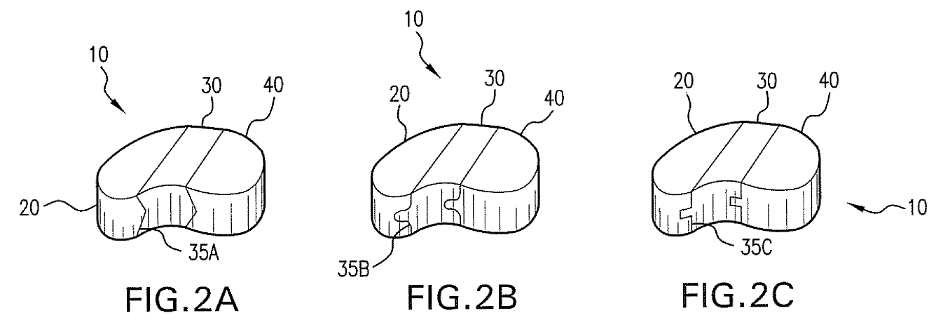

FIGS. 2A, 2B, and 2C illustrate alternative embodiments of the implant of this invention. In FIG. 2A, the implant 10 is composed of three components 20, 30, and 40, where the implant is configured such that the components combine to form the implant and where the components abut along sides that include two angled surfaces. In this case, the angled surfaces form a V-shape when viewed from the side of the implant. The slant and length of each edge of the V-shape can vary widely.

In FIG. 2B the implant 10 includes components that form a tongue-and-groove type abutment 35B between components where the tongue has a rounded shape. FIG. 2C also shows an implant 10 with a tongue and groove type abutment, where the tongue has sharp edges, and is not rounded at the portion that protrudes furthest into the complimentary component.

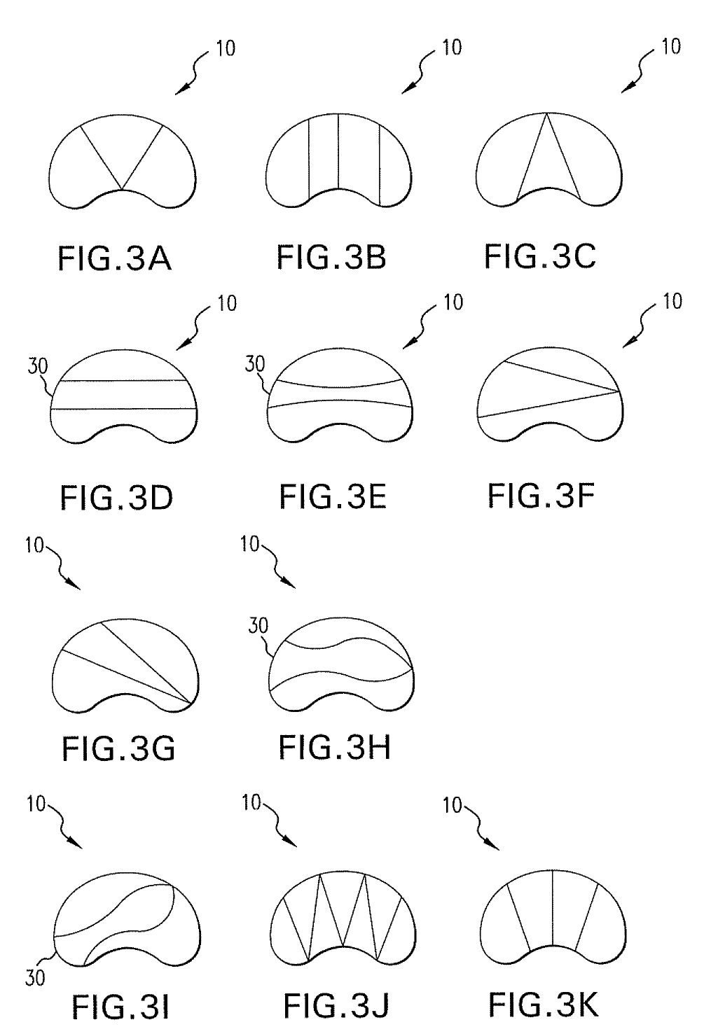

FIGS. 3A through 3K illustrate alternative shapes and configurations of the components from a top view of the implant. The implant can include any number of components. In FIG. 3A there are three components whereas four components are used in FIG. 3K and seven components are used in FIG. 3J. In FIGS. 3A, 3C, 3F, and 3G the second component 30 is pie shaped (generally triangular or “wedge” shaped), with straight edges where the components abut. In FIGS. 3B and 3K, four components sit side-by-side, with each side that abuts another component having a straight edge. In FIG. 3K, the edges of the components are not parallel to the other edges, as the edges are arranged in FIG. 3B. In FIG. 3D, a three-component implant 10 is depicted in which the second component 30 is aligned in a direction perpendicular to the posterior-anterior direction of a disc. The implant 10 of FIG. 3E is also aligned in such a perpendicular direction except the second component 30 has curved edges that abut the other components, unlike the straight edges in FIG. 3D. FIGS. 3H and 3I illustrate implants 10 with a fish-shaped second component 30, with the implants 10 having different configurations for the second component 30, with the implant 10 of FIG. 3H arranged in a direction generally perpendicular to the anterior-posterior line of a disc, and the implant in FIG. 3I arranged in a direction at offset relative to the anterior-posterior line. In FIG. 3J, a seven-component implant 10 is shown where each component has a triangular or wedge shape as viewed from the top.

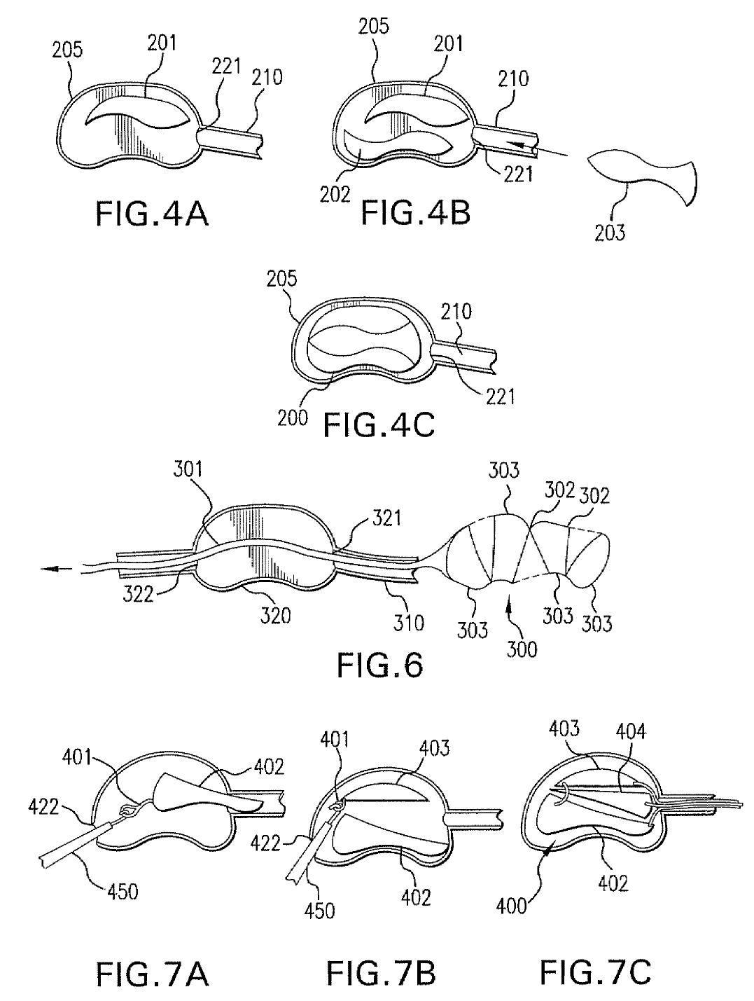

FIGS. 4A-4C generally illustrate the introduction of implant components 201, 202, and 203 in the formation of implant 200 within a disc 205. Thus, a first component 201 is introduced through a cannula 210 and hole (or “port”) 221 in the annulus 220 that has been created by the physician. After a second component 202 is introduced into the disc 205 as in FIG. 4B, the third component 203 is inserted. The third component 203 can be configured so that the front edge or tip pushes components 201, 202 out of the way while the third component 203 is being inserted. The components together form the implant 200 as depicted in FIG. 4C. It should be appreciated that the particular sequence and method of creating the spinal implant within the disc will vary depending on the configuration of the implant.

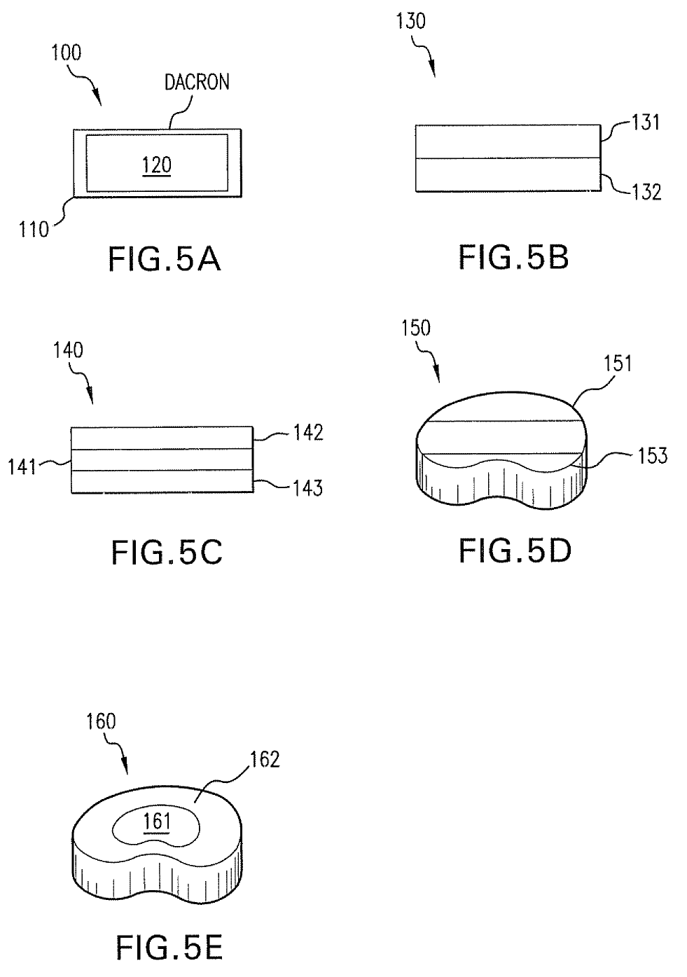

FIG. 5A illustrates a cross-section of another embodiment of this invention. In this embodiment 100, an outer layer (jacket) 110 of Dacron material, for example, surrounds the viscoelastic inner core 120. This Dacron jacket can facilitate bone growth. The outer Dacron layer may be employed in any of the embodiments of this invention.

FIGS. 5B and 5C show a two layer implant 130 and a three layer implant 140. Each of the layers in implants 130, 140 can be made of different materials such as made of different polymers, or made of the same polymers of different properties such as having different hardness, wear ability, and so on. For example, in implant 140 a soft middle layer 141 can be sandwiched between layers 142, 143 that provide improved wear resistance (tougher) at the points in contact with a portion of a body where abrasion occurs, relative to the softer middle layer 141. Similarly, in implant 130, a first layer 131 can be made of a different polymer than the second layer 132.

In FIG. 5D, sides 151 and 153 of implant 150 may be composed of a tougher material. Implant 150 may be advantageous because it is known that in a given joint the weight distribution during use can vary, with the anterior and posterior portions of a disc carrying more load than the middle portion of the disc. The configuration of implant 150 can be adjusted to correspond to the load characteristics of a given disc. The implant 160 of FIG. 5E provides similar load bearing characteristics to that in FIG. 5D except that an inner core 161 is surrounded on all sides by an outer layer 162 made of a tougher, more wear resistant material. Through use of the embodiments in FIGS. 5D and 5E it may be possible to extend the useful life of the implant.

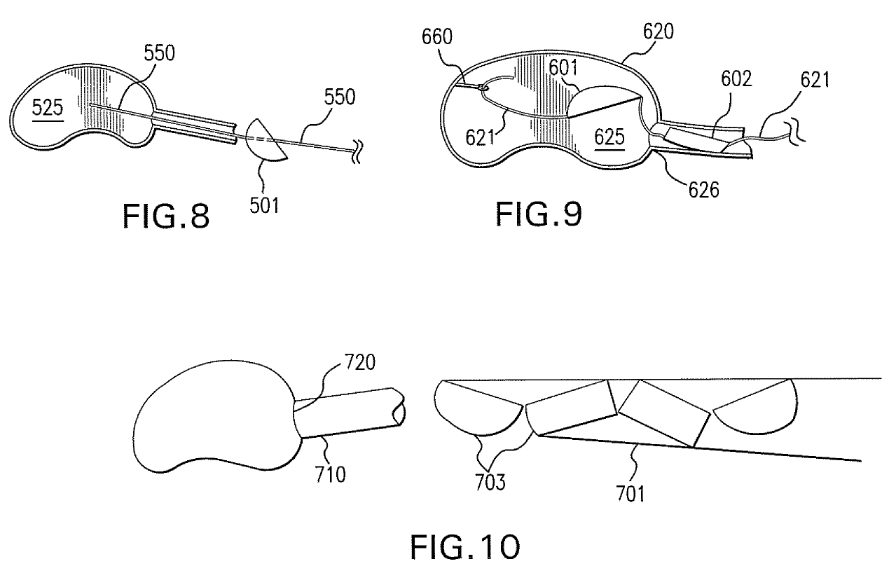

FIG. 6 shows another embodiment of this invention, where the implant 300 is inserted through a cannula 310 and a first hole 321 in the annulus 320 by inserting a link (e.g., thread) 301 attached to the implant 300, and drawing the link 301 through the disc and out a second hole 322 in the annulus 320 through use of a suitable instrument that can grab the end of the thread. The link may also be referred to herein as a coupling element, connective element, or connective member. Alternatively, only one hole is made in the annulus for introduction of implant 300. In this embodiment, the implant 300 is anchored either internally or externally to the annulus on the side opposite the hole in the annulus, such as through use of a dart, barb, pin, suture, or other type of anchor. The implant 300 is configured so that the link 301 attaches to portions 302 of the implant, and configured such that the components 303 of the implant are attached at other positions. The implant 300 is configured so that proximal cinching of the link(s) brings the components 303 into place within the disc to form the final implant. The implant can be sutured to hold its configuration. In one embodiment, implant 300 as well as other implant embodiments herein, can be adapted to include an anchor that attaches to the side of the annulus opposite the hole in the annulus. For example, in FIG. 9 there is shown components 601, 602 that are joined by link 621 that also has been inserted through an anchor 660 that has been secured in annulus 620 within disc space 625, wherein the anchor 660 is positioned opposite the port 626 in the annulus 620. Alternatively, the implant is adapted for use with a separate anchor that internally or externally anchors to the annulus. In addition, implant 300 or other implant embodiment herein can be guided into the disc space through use of a guidance member, such as a Kirschner wire (i.e., K-wire), that allows the guidance member to be positioned in the disc space, components slid down the guidance member and into the disc space via holes for example in the components, with the guidance member then used to position the components and optionally anchor the components and then removed from the disc space. Thus FIG. 8 illustrates an implant component 501 is positioned in the disc space 525 using a guidance member 550.

FIGS. 7A-7C show an embodiment similar to that depicted in FIG. 6 whereby the components 402, 403, and 404 are pulled into place using an instrument 450 that can grab a thread 401 through a second hole 422 in the annulus 420. The components 402, 403, and 404 together form the implant 400, which can be stitched together as shown in FIG. 6C.

FIG. 10 shows another embodiment of an implant 700 inserted through a cannula 710 and a hole 720 through the annulus. The implant 700 has components 703 that are aligned in the disc space by a proximal cinching of links 705 and 706. The links may be tied and/or anchored to keep the components aligned.

The spinal implant of this invention can be made from a variety of materials that provide viscoelastic properties. For example, elastomeric polyurethanes well known to those of skill in the art can be used. Likewise, hydrogels, various silicones, and combinations of materials can be used.

The polymers that can be used in the practice of this invention to make the polymerizable compositions and polymerized elastic materials (including hydrogels) include but are not limited to polyacrylonitrile, polyvinyl alcohol, polyvinylpyrrolidone, polyacrylic acid, polymethacrylic acid, polyurethane, polyurea, polytetrafluoroethylene, cellulose triacetate, polydimethylsiloxane, polyacrylamide, polyethyleneoxide, copolymers of ethylene oxide and propylene oxide or hyaluronic acid, (pliable) epoxy polymers, and combinations thereof, as well as the monomers used to make such polymers. The monomers that can be employed to make the polymers used in this invention include but are not limited to hydroxyalkyl acrylates such as 2-hydroxy ethyl methacrylate, acrylic acid, acrylonitrile, urea, ethylene oxide and propylene oxide, acrylamide, tetrafluoroethylene, dimethylsiloxane, monomers used to form polyurethane such as polyols and diisocyanates such as diphenylmethane diisocyanate (MDI), monomers used to form pliable epoxy resins, vinyl alcohol, methacrylates including alkyl methacrylates such as methyl methacrylate, N-vinyl monomers such as N-vinyl-2-pyrrolidone, ethylenically unsaturated acids such as methacrylic acid, ethylenically unsaturated bases such as 2-(diethylamino) ethyl methacrylate. The polymers can be made using well known techniques and are available commercially. Likewise, polymers can be readily formed into sheets and so on, as described herein, using well known techniques.

In general, if monomers and/or polymer precursors are introduced into the cavity, the monomers and/or polymer precursors react in the body to form the final polymeric composition. As used herein, “polymer precursor” (which can also be referred to as a “prepolymer”) refers to materials that are formed by the partial polymerization of monomers, such as to form chains by reaction of, for example, two to four monomer groups.

In some cases, depending on the type of monomers or polymer precursors employed, polymerization initiators or catalysts are required to cause polymerization. Such compounds can be, for example, free radical initiators. In other cases, heat or light (e.g., UV light) can serve to initiate polymerization.

Representative examples of suitable polymeric materials are described in U.S. Pat. No. 5,976,186, U.S. Pat. No. 6,264,695, U.S. Pat. No. 6,280,475, U.S. Pat. No. 6,443,988, and U.S. Pat. No. 6,595,998, each of which is incorporated herein by reference in their entirety.

The implant can be introduced as an at least partially dehydrated solid. In this regard, the at least partially dehydrated solid becomes re-hydrated after being introduced. The elastic material can thus swell to a larger size than the incision or hole that the elastic material is introduced through, thereby preventing the swelled elastic material from undesirably becoming expelled from the disc. Beneficially, the material can be readily removed for replacement or if another procedure is required.

The implants can be made in a variety of ways. For example, a body can be made using techniques such as injection molding. This body can then be cut into the desired number of components, in the desired shapes and signs. Likewise, individual molds can be made to independently form a plurality of components whose shapes and sizes mate to form the spinal implant. The techniques used to form the components from molds are well known to those of skill in the art. In general, a polymerizable composition is introduced into the mold, the composition polymerizes, and the resulting hardened component is removed. Alternatively, for example, a thermoplastic material is melted, introduced into the mold, and then allowed to cool and solidify. The solid compound is then removed from the mold. Other methods well known to those of skill in the art can be used.

The implants of this invention are viscoelastic, or more generally elastomeric. It should be appreciated that an implant can be made from two or more materials, each having different elasticity. Likewise, the implant can be a composite, or a uniform body formed from blends of elastomeric materials. Likewise, the implant can include a variety of additives such as antibiotics, growth factors (tissue, bone, etc.) radiopaque agents, anti-inflammatory agents, and compounds to make the implant slick during the procedure to introduce the implant into the disc. The additives can be embedded in the implant (blended in), sprayed onto the implant, or both. It may be desirable to permanently mark the implant, such as with a barium or tantalum marker, so that the implant position can be confirmed. Alternatively, the implant can include a radiopaque layer or particles.

The implant can include a barrier layer or coating such as a hydrogel that sloughs away during use to thereby reduce scarring and scar accumulation. The coating can also serve as an adhesion barrier. Materials used to form the barrier layer or coating are well known, including but not limited to polyethylene glycol (PEG), polylactide, polyglycolic acid, collagen, carboxymethylcellulose, hyaluronic acid, and cellulose.

The spinal implants of this invention can be inserted into a disk through a variety of methods. In general, the implant is introduced through a posterior approach, including an extreme lateral approach, a posterior approach, and a transforaminal approach. Prior to insertion of the implant, a discectomy is performed to remove nucleus pulposus using well known techniques. A hole in the annulus can be created in the annulus through use of an endoscope, or similarly functional instrument. Other instruments can also be used to dock with the annulus for insertion of the implant. The discectomy can optionally be performed using the endoscope. The components of the spinal implant are inserted (pushed) through the endoscope or a cannula to position the components into the disc. Optionally, a second hole may be established to allow components to be both pushed and pulled into position. In general, the internal diameter of the endoscope or cannula will be the limiting factor for the size of the implant components being inserted into the disc.

After the implant has been placed within the disc, the annulus can be repaired to close the hole. The annulus can be closed using, for example, sutures or a biocompatible glue. An annulus repair kit can be employed in this regard. Depending on the size of the hole or other circumstances, it may not be necessary to close the hole.

The implant is configured depending on the size of the disc space. Thus, the height and diameter of the disc can be adjusted depending on which disc in the patient is being treated. In general, the implant of this invention has a height of from about 1 to about 15 mm. The width and shape of the implant are dictated by the disc.

In some cases it may be necessary to subject the disc space to distraction prior to insertion of an implant. The distraction can be conducted using well known procedures and instrumentation.

Further modifications and alternative embodiments of this invention will be apparent to those skilled in the art in view of this description. Accordingly, this description is to be construed as illustrative only and is for the purpose of teaching those skilled in the art the manner of carrying out the invention. It is to be understood that the forms of the invention herein shown and described are to be taken as illustrative embodiments. Equivalent elements or materials may be substituted for those illustrated and described herein, and certain features of the invention may be utilized independently of the use of other features, all as would be apparent to one skilled in the art after having the benefit of this description of the invention.

What is claimed is:

1. A multicomponent spinal implant, comprising: a plurality of components adapted for use as an implant in a disc space, wherein each component is configured to be inserted into the disc space and wherein the components interconnect within the disc space to form the spinal implant.

2. The spinal implant of claim 1, wherein each component complimentarily abuts at least one other component to form the implant.

3. The spinal implant of claim 1, wherein the components are interconnected by at least one link that is adapted to facilitate cinching of the components within the disc space.

4. The spinal implant of claim 1, wherein the implant is adapted to be anchored to an annulus on a side opposite to the side where the implant is introduced into the disc space.

5. The spinal implant of claim 1, wherein a first component has a convex side surface and a second component has a concave side surface, which compliments the convex side of the first component.

6. The spinal implant of claim 1, wherein at least two components have complimentary flared sides to reduce motion when in the disc space.

7. The spinal implant of claim 1, wherein the components are shaped to pair together so as to limit range of motion relative to one another in a direction generally parallel to the spine.

8. The spinal implant of claim 1, wherein the implant comprises three components.

9. The spinal implant of claim 1, comprising from 2 to 6 components.

10. The spinal implant of claim 1, wherein each component has a top, bottom, and at least one side, wherein the side has a surface generally perpendicular to the surfaces of the top and bottom.

11. The spinal implant of claim 1, wherein each component has a top, bottom, and at least one side, wherein the side has a surface that is not perpendicular to the surfaces of the top and bottom.

12. The spinal implant of claim 1, wherein the implant comprises three components that align in a void in a disc after a discectomy to generally form a shape that substantially fills the void.

13. The spinal implant of claim 1, wherein the implant is made of at least one synthetic elastic material.

14. The spinal implant of claim 1, wherein at least one component includes a radiopaque agent.

15. The spinal implant of claim 1, wherein the components are jacketed with a material that facilitates bone growth.

16. The spinal implant of claim 1, wherein at least one portion of the perimeter of the implant is made of material that has greater wear resistance than other portions of the implant.

17. A method of treating a spinal disc, comprising:
creating a port from a posterior approach into a disc space of a spinal disc; and
inserting components that form a spinal implant into the disc space through the port to form the spinal implant.

18. The method of claim 17, further comprising repairing the tissue through which the port has been created.

19. The method of claim 17, wherein the implant comprises a plurality of components adapted for use as an implant in a disc, wherein each component is configured to be inserted into the disc space, wherein each component complimentarily abuts at least one other component to form the implant.

20. The method of claim 17, wherein a first component has a convex side surface and a second component has a concave side surface, which compliments the convex side of the first component.

21. The method of claim 17, wherein at least two components have complimentary flared sides to reduce motion when in the disc.

22. The method of claim 17, wherein the components are shaped to pair together so as to limit range of motion relative to one another in a direction generally parallel to the spine.

23. The method of claim 17, wherein the implant comprises three components.

24. The method of claim 17, wherein the implant comprises from 2 to 6 components.

25. The method of claim 17, wherein each component has a top, bottom, and at least one side, wherein the side has a surface generally perpendicular to the surfaces of the top and bottom.

26. The method of claim 17, wherein each component has a top, bottom, and at least one side, wherein the side has a surface that is not perpendicular to the surfaces of the top and bottom.

27. The method of claim 17, wherein the implant comprises three components that align in a void in a disc after a discectomy to generally form a shape that substantially fills the void.

28. The method of claim 17, wherein the implant is made of at least one synthetic elastic material.

29. The method of claim 17, wherein at least one component includes a radiopaque agent.

30. The method of claim 17, wherein the components are jacketed with a material that facilitates bone growth.

31. The method of claim 17, wherein at least one portion of the perimeter of the implant is made of material that has greater wear resistance than other portions of the implant.

32. The method of claim 17, wherein the components are inserted using a guidance member.

33. The method of claim 17, wherein the components include a link that is cinched proximally to interconnect the components and form the spinal implant.

34. The method of claim 17, wherein at least one component is anchored to a wall of the annulus opposite the port.

35. A method of forming a modular spinal implant, comprising: preparing a plurality of components that are shaped for insertion into a disc space, wherein each component abuts at least one other component in the disc space.

36. The method of claim 35, wherein the components are interconnected by at least one link that is adapted to facilitate aligning of the components within the disc space.

37. The method of claim 35, wherein the implant comprises a plurality of components adapted for use as an implant in a disc space, wherein each component is configured to be inserted into the disc space, wherein each component complimentarily abuts at least one other component to form the implant.

38. The method of claim 35, wherein a first component has a convex side surface and a second component has a concave side surface which compliments the convex side of the first component.

39. The method of claim 35, wherein at least two components have complimentary flared sides to reduce motion when in the disc space.

40. The method of claim 35, wherein the components are shaped to pair together so as to limit range of motion relative to one another in a direction generally parallel to the spine.

41. The method of claim 35, wherein the implant comprises three components.

42. The method of claim 35, wherein the implant comprises from 2 to 6 components.

43. The method of claim 35, wherein each component has a top, bottom, and at least one side, wherein the side has a surface generally perpendicular to the surfaces of the top and bottom.

44. The method of claim 35, wherein each component has a top, bottom, and at least one side, wherein the side has a surface that is not perpendicular to the surfaces of the top and bottom.

45. The method of claim 35, wherein the implant comprises three components that align in a void in a disc after a discectomy to generally form a shape that substantially fills the void.

46. The method of claim 35, wherein the implant is made of at least one synthetic elastic material.

47. The method of claim 30, wherein at least one component includes a radiopaque agent.

48. The method of claim 30, wherein the components are jacketed with a material that facilitates bone growth.

49. The method of claim 30, wherein at least one portion of the perimeter of the implant is made of material that has greater wear resistance than other portions of the implant.

50. A multicomponent spinal implant, comprising: a plurality of components adapted for use as an implant in a disc space, wherein each component is configured to be inserted into the disc space, wherein the components interconnect within the disc to form the spinal implant, and wherein the components are interconnected by at least one link that is adapted to facilitate cinching of the components within the disc.

51. The spinal implant of claim 49, wherein each component complimentarily abuts at least one other component to form the implant.

52. The spinal implant of claim 49, wherein the implant is adapted to be anchored to an annulus on a side opposite to the side where the implant is introduced into the disc.

53. The spinal implant of claim 49, wherein a first component has a convex side surface and a second component has a concave side surface, which compliments the convex side of the first component.

54. The spinal implant of claim 49, wherein at least two components have complimentary flared sides to reduce motion when in the disc.

55. The spinal implant of claim 49, wherein the components are shaped to pair together so as to limit range of motion relative to one another in a direction generally parallel to the spine.

56. The spinal implant of claim 49, wherein the implant comprises three components.

57. The spinal implant of claim 49, comprising from 2 to 6 components.

58. The spinal implant of claim 49, wherein each component has a top, bottom, and at least one side, wherein the side has a surface generally perpendicular to the surfaces of the top and bottom.

59. The spinal implant of claim 49, wherein each component has a top, bottom, and at least one side, wherein the side has a surface that is not perpendicular to the surfaces of the top and bottom.

60. The spinal implant of claim 49, wherein the implant comprises three components that align in a void in a disc after a discectomy to generally form a shape that substantially fills the void.

61. The spinal implant of claim 49, wherein the implant is made of at least one synthetic elastic material.

62. The spinal implant of claim 49, wherein at least one component includes a radiopaque agent.

63. The spinal implant of claim 49, wherein the components are jacketed with a material that facilitates bone growth.

64. The spinal implant of claim 49, wherein at least one portion of the perimeter of the implant is made of material that has greater wear resistance than other portions of the implant.

65. The spinal implant of claim 49, wherein each component has at least one hole adapted for receipt of a guidance member.
